(19) United States
(12) Plant Patent Application Publication
(10) Pub. No.: US 2002/0104138 P1
(43) Pub. Date: Aug. 1, 2002

(54) Agapanthus orientalis named 'Glen Avon'

(76) Inventor: Alan D. Gray, Taranaki (NZ)

Correspondence Address: KLARQUIST SPARKMAN CAMPBELL LEIGH & WHINSTON, LLP, One World Trade Center, Suite 1600, 121 S.W. Salmon Street, Portland, OR, 97204, US

(21) Appl. No.: 09/772,464

(22) Filed: Jan. 29, 2001

(51) Int. Cl. A01H 5/00

(52) U.S. Cl. PLT/263

(57) ABSTRACT

A new and distinct selection of Lily of the Nile, which is denominated as Agapanthus orientalis named ‘Glen Avon’.

### BACKGROUND

This invention relates to a new and distinct selection of Agapanthus orientalis which is native to Southern Africa and a member of the amaryllis family. The new variety, Agapanthus orientalis cv. ‘Glen Avon’ was the result of a breeding program. The pollen parent is unknown. The new variety is from a cross made in 1987, and seed was sown in 1988. The first year of flowering was in 1991, and the original plant of the new ‘Glen Avon’ variety was selected from the seedlings in 1994 which were being grown at New Plymouth, Taranaki, New Zealand. In 1995, the first initiation into tissue culture was made at Lifetech Laboratories Ltd. located in Albany, Auckland, New Zealand.

The new plant has been asexually reproduced by tissue culture since its discovery with such reproduction being performed at Lifetech Laboratories location in Auckland, New Zealand. Through extensive propagation through tissue culture, it has been established that the plant is true to type and the novel exceptional characteristics of this new plant are stable and reliably passed on to progeny through asexual reproduction.

### SUMMARY

Agapanthus orientalis cv. ‘Glen Avon’ possesses extremely dense inflorescence. The quantity of florets is almost double that of a typical Agapanthus orientalis inflorescence. Due to the large number of florets, the inflorescence typically exhibits a solid, rounded, compact ball of 20 centimeters or more in diameter.

The petal color is a medium lilac-blue with the center midrib a darker lilac-blue, exhibiting a two-toned effect in each floret.

The perianth in Agapanthus orientalis is generally six (6) parted. Many of the flowers of the ‘Glen Avon’ selection exhibit a perianth that is six (6) to eleven (11) parted, producing a semi-double to double flower effect. The cultivar ‘Glen Avon’ has tolerated temperatures as low as 10° F. and as high as 40° F. This is equivalent to an USDA hardiness zone of eight (8) to eleven (11).

It should be noted that growth and/or color varies with time of year, lighting conditions, and soil and nutrient conditions. For example, leaf colors may be brighter green if the plant is grown in soil with greater nitrogen concentrations, and may be more yellow when grown in soil containing lesser amounts of nitrogen.

### BRIEF DESCRIPTION OF THE DRAWING

FIG. 1 is a photograph showing an entire ‘Glen Avon’ plant in flower.

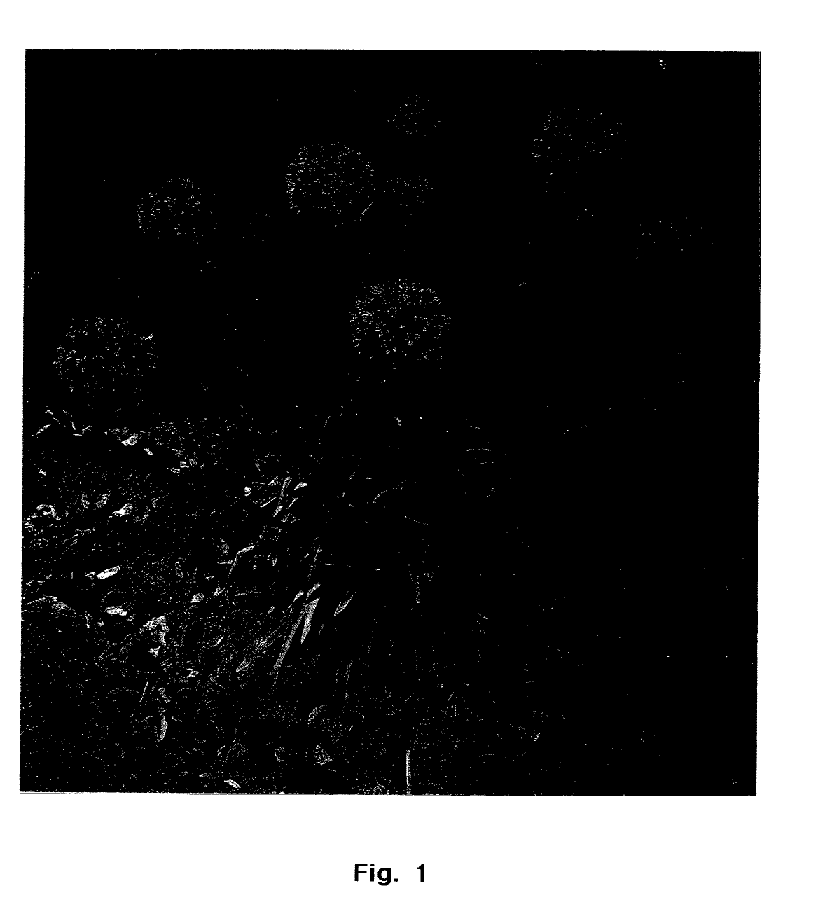

FIG. 2 is a photograph showing a close up of flowers produced by the cultivar ‘Glen Avon’. FIG. 2 shows the inflorescence exhibiting the extra parted perianth segments, the darker midribs and the dense rounded head.

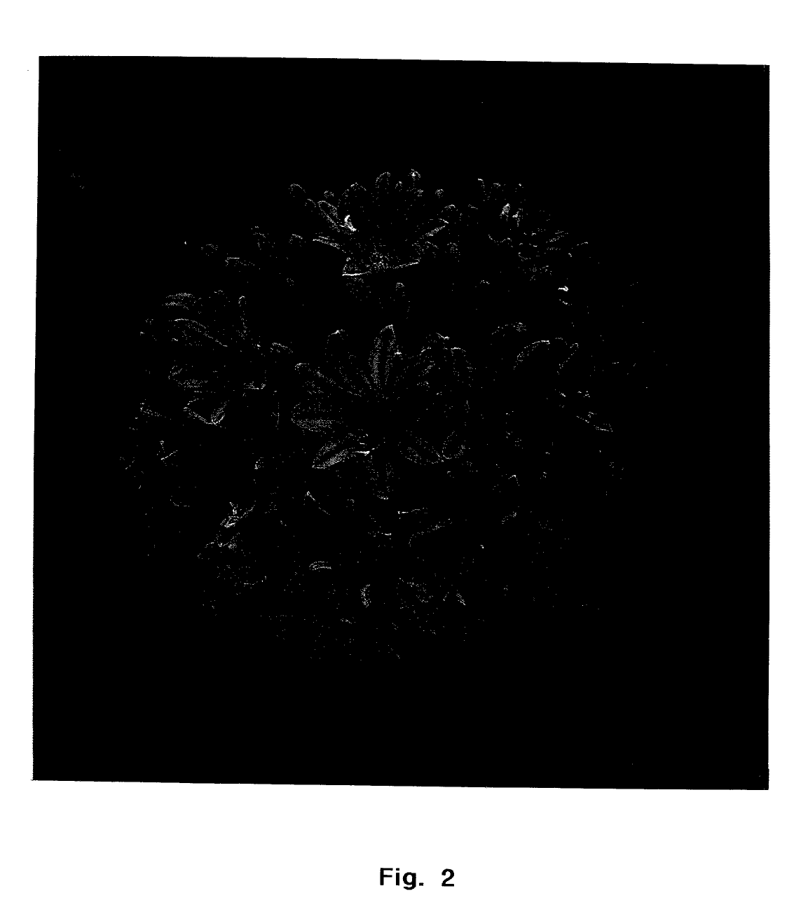

### BOTANICAL DESCRIPTION

Below is a detailed description of Agapanthus orientalis cultivar ‘Glen Avon’. The color terminology is from The Royal Horticultural Society Color Charts. Measurements are based on plants grown in Australia and also in California.

Overall habit: Herbaceous perennial.

Overall size: Maximum observed height from soil surface to top of inflorescence as of this time ranges from 90 cm to 120 cm with a typical height range of 100 cm to 110 cm. Maximum height from soil surface to upper most of leaves at flowering time typically ranges to 50 cm tall.

Bloom Season in California: Bloom commences in early summer and continues up to about three months, thus, throughout summer months ending in late summer.

Foliage: linear, strap-like.

Size.—Length: From base to up of mature foliage, typically longer than 40 cm and generally shorter than 70 cm. Width: Maximum width of mature foliage, typically wider than 4.0 cm, generally 5.0 cm.

Number.—Leaves per bud at flowering time, typically more than eleven (11).

Shape of Leaf Tip.—Obtuse to slightly acute.

Margins.—Smooth, entire.

Color.—Upper leaf surface, Like Yellow-Green 147B. Lower leaf surface, Like Yellow-Green 147B.

Texture.—Slightly fleshy, succulent.

Inflorescence:

Arrangement.—Single umbel, no secondary inflorescence.

Width.—Maximum width at time of flowering typically wider than 20 cm, on average 22 cm.

Height.—Side view at time of flowering typically ranging from 10 cm to 20 cm, on average 13 cm.

Flower:

Scape.—Length: From soil surface to base of inflorescence at the time of flowering typically over 80 cm, averaging 95 cm. Thickness: Typical maximum thickness at the middle point of the scape at the time of flowering averaging 1.7 cm. Cross Section: Shape at middle point of scape at time of flowering—elliptic. Angle: At time of flowering—straight. Color: At middle portion of the scape at time of flowering: Like Yellow-Green Group 147.

Florets:

Perianth shape.—Open, wide, spreading.

Number of Perianth Segments.—Total typically six (6) to eleven (11) segments, averaging eight (8).

Double perianth.—Perianth color: Inner: Like Violet-Blue Group 91 B, C, D with center inner midrib like 91A. Outer: Like Violet-Blue Group 91C on lower bottom half, like 91B with tint of Violet-Blue Like Group 91A, also like Violet-Blue Group 91A along outside midrib. Mature: As flowers age they darken to a like Violet-Blue Group 92A.

Number of Florets per Inflorescence: Typically more than 200 and ranging from 200-300.

Florets.—Length: Side view from base to tip of floret, typically 4.0 cm to 4.5 cm. Width: Diameter typically averaging 3 0 cm.

Pedicel.—Length: 6-7 cm long. Color: Like Yellow-Green 146C and 146D.

Stamens.—Number: Eight (8). Arrangement: Adhere to within throat typically 1.6 cm from base, 2 cm long from perianth. Anthers: Typically 3 cm in length Color: Like Yellow-Green 154D. Pollen color: Like Yellow-Green 154C.

Pistol.—Length of style: Typically averages 17 mm long.

Fruit.—Aloculicidal capsule, 4 angled. Size: Typically 3.5 cm long×1.51 cm wide. Production: Only a few fruit are produced. Thirteen (13) fruit from 250 flowers, less than 6% if the florets in these observations produced fruit.

Disease resistance: As is typical of this species, the ‘Glen Avon’ cultivar exhibited a strong disease resistance under normal growing conditions.

I claim:

1. A new and distinct Agapanthus orientalis plant named ‘Glen Avon’ substantially as shown and described which differs distinctively from other Agapanthus orientalis plants by its unique combination of soft lilac florets with up to eleven petals per floret, flower heads composed of an abundant number of individual florets, a low percentage of which produce fruit and a prolonged flowering period.
